(19) United States
(12) Patent Application Publication
(10) Pub. No.: US 2025/0084444 A1
(43) Pub. Date: Mar. 13, 2025

(54) FERMENTATION METHODS USING ACETOGENIC CARBOXYDOTROPHIC BACTERIA

(71) Applicant: SYNATA BIO, INC., Warrenville, IL (US)

(72) Inventors: David KEATING, Warrenville, IL (US); Jennine B. TERRILL, Warrenville, IL (US)

(73) Assignee: SYNATA BIO, INC., Warrenville, IL (US)

(21) Appl. No.: 18/728,387

(22) PCT Filed: Jan. 11, 2023
(86) PCT No.: PCT/US2023/060498

§ 371 Date: Jul. 11, 2024

Related U.S. Application Data
(60) Provisional application No. 63/369,683, filed on Jul. 28, 2022.

(51) Int. Cl. C12P 7/06; C07K 14/33; C12N 1/20; C12N 9/10; C12N 9/88; C12R 1/145

(52) U.S. Cl. CPC C12P 7/065; C07K 14/33; C12N 1/20; C12N 9/13; C12N 9/88; C12R 2001/145; C12Y 207/07073; C12Y 208/0101; C12Y 401/99019

(57) ABSTRACT

Disclosed are methods for producing from a gaseous substrate comprising CO, CO₂, and optionally H₂ at least one oxygenated product. Also provided are methods of using a broth deficient in thiamine or thiazole-containing thiamine precursors as a screening tool for suitable ethanol producing acetogenic carboxydotrophic bacteria strains, operating a syngas fermentation without the addition of thiamine or thiazole-containing thiamine precursors, controlling bacterial contamination in a bioreactor, and methods for preparing animal feed and fertilizer.

### CROSS REFERENCE TO RELATED APPLICATIONS

This patent application claims the benefit of co-pending U.S. Provisional Patent Application No. 63/369,683, filed Jul. 28, 2022, which is incorporated by reference herein in its entirety.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 7,154 Byte XML file named “766308.xml,” created Jan. 5, 2023.

### BACKGROUND OF THE INVENTION

It is desirable to use bacteria to convert gases containing carbon monoxide (CO) and carbon dioxide (CO₂), and optionally hydrogen (H₂), such as industrial waste gas or syngas, into a variety of products, such as fuels and chemicals using fermentation. Acetogenic bacteria that can consume CO, CO₂, and H₂ have been shown to require vitamin supplementation, with pantothenate (Vitamin B5), thiamine (Vitamin B1), and biotin (Vitamin B7) for growth (Annan, et al., Appl. Microbiol. Biotechnol., 103: 4633-4648 (2019)). Providing the vitamin supplements to the bacteria during large batch fermentations can be costly. Further, removal of vitamin supplements reduces opportunities for growth of contaminating microorganisms.

Accordingly, there remains a need for methods that reduce the need for vitamin supplementation associated with continuous fermentation. Further, there is a need for reducing contamination in continuous fermentations.

### BRIEF SUMMARY OF THE INVENTION

An embodiment of the invention provides methods for producing from a gaseous substrate comprising CO, CO₂, and optionally H₂ at least one oxygenated product, the method comprising: (a) providing to a bioreactor: (1) a gaseous substrate comprising CO, CO₂, and optionally H₂, (2) at least one acetogenic carboxydotrophic bacterium, and (3) a liquid nutrient medium, and (b) providing conditions within the bioreactor for the at least one acetogenic carboxydotrophic bacterium to convert the gaseous substrate to at least one oxygenated product, wherein the at least one acetogenic carboxydotrophic bacterium comprises at least one gene selected from thiH, thiS, thiF, and thiG.

Another embodiment of the invention provides methods for producing from a gaseous substrate comprising CO, CO₂, and optionally H₂ at least one oxygenated product, the method comprising: (a) providing to a bioreactor: (1) a gaseous substrate comprising CO, CO₂, and optionally H₂, (2) at least one acetogenic carboxydotrophic bacterium, and (3) a liquid nutrient medium, and (b) providing conditions within the bioreactor for the at least one acetogenic carboxydotrophic bacterium to convert the gaseous substrate to at least one oxygenated product, wherein the at least one acetogenic carboxydotrophic bacterium does not require exogenously supplied thiamine or thiazole-containing thiamine precursors.

A further embodiment of the invention provides methods of operating a syngas fermentation without the addition of thiamine or thiazole-containing thiamine precursors, the method comprising: (a) providing to a bioreactor: (1) a gaseous substrate comprising CO, CO₂, and optionally H₂, (2) at least one acetogenic carboxydotrophic bacterium, and (3) a liquid nutrient medium that does not contain thiamine or thiazole-containing thiamine precursors, and (b) providing conditions within the bioreactor for the at least one acetogenic carboxydotrophic bacterium to convert the gaseous substrate to at least one oxygenated product, wherein the conditions within the bioreactor create a bioreactor broth, and thiamine or thiazole-containing thiamine precursors is not added to (i) the liquid nutrient medium or (ii) the bioreactor broth before or during syngas fermentation.

An additional embodiment of the invention provides methods for producing from a gaseous substrate comprising CO, CO₂, and optionally H₂ at least one oxygenated product, the method comprising (a) providing to a bioreactor: (1) a gaseous substrate comprising CO, CO₂, and optionally H₂, (2) at least one acetogenic carboxydotrophic bacterium, and (3) a liquid nutrient medium, and (b) providing conditions within the bioreactor for the at least one acetogenic carboxydotrophic bacterium to convert the gaseous substrate to at least one oxygenated product, wherein the at least one acetogenic carboxydotrophic bacterium comprises at least one gene (e.g., an exogenous gene) selected from thiH, thiS, thiF, and thiG.

Another embodiment of the invention provides methods of using broth deficient in thiamine or thiazole-containing thiamine precursors as a screening tool for suitable ethanol producing acetogenic carboxydotrophic bacteria strains, the method comprising: (a) providing to a bioreactor in the absence of thiamine or thiazole-containing thiamine precursors: (1) a gaseous substrate comprising CO, CO₂, and optionally H₂, (2) at least one acetogenic carboxydotrophic bacterium, and (3) a liquid nutrient medium, and (b) providing conditions within the bioreactor for the at least one acetogenic carboxydotrophic bacterium to convert the gaseous substrate to at least one oxygenated product, wherein the conversion of the gaseous substrate to at least one oxygenated product indicates the presence of a suitable ethanol producing acetogenic carboxydotrophic bacteria strain.

A further embodiment of the invention provides methods of controlling bacterial contamination in a bioreactor, the method comprising: (a) providing to a bioreactor in the absence of thiamine or thiazole-containing thiamine precursors: (1) a gaseous substrate comprising CO, CO₂, and optionally H₂, (2) at least one acetogenic carboxydotrophic bacterium, and (3) a liquid nutrient medium, and (b) providing conditions within the bioreactor for the at least one acetogenic carboxydotrophic bacterium to convert the gaseous substrate to at least one oxygenated product, wherein the absence of exogenously supplied thiamine or thiazole-containing thiamine precursors in the bioreactor prevents contamination of the bioreactor broth with non-desirable acetogenic carboxydotrophic bacteria.

Additional aspects include methods for preparing animal feed and for preparing fertilizer.

### BRIEF DESCRIPTION OF THE SEVERAL VIEWS OF THE DRAWING(S)

FIG. 1 is a schematic of the 11-step pathway involved in thiamine production.

FIG. 2 is a graph showing H₂, CO, and CO₂ uptake of acetogenic carboxydotrophic bacteria strain SB1 (an aspect of the invention) during fermentation after removal of thiamine from the growth medium. The black arrow indicates when thiamine was removed from the growth medium.

FIG. 3 is a graph showing ethanol and acetate titers produced by acetogenic carboxydotrophic bacteria strain SB1 during fermentation after removal of thiamine from the growth medium. The black arrow indicates when thiamine was removed from the growth medium.

FIG. 4A is a schematic of a plasmid construct used for single cross-over gene disruption in accordance with an aspect of the invention. The serrated regions to left of thiS and to the right of thiF indicate that 5′ and 3 regions are missing for thiS and thiF, respectively.

FIG. 4B is an image showing a closeup of colonies present on a representative plate showing robust growth in thiamine containing medium in accordance with an aspect of the invention.

FIG. 4C is an image of PCR gel showing the presence of integration in the region of thiHSFG in accordance with an aspect of the invention. Numbers 1-5 represent the 5 colonies tested.

FIG. 4D is an image showing the growth of cells in media using fructose as a carbon source in the absence “−” or presence “+” of thiamine (left). The graph on right shows OD values for the cultures grown in the absence of presence of thiamine. Taken together, FIGS. 4A-4D show inactivation of SEQ ID NO: 1 (thiH), SEQ ID NO: 2 (thiS), SEQ ID NO: 3 (thiF), and SEQ ID NO: 4 (thiG) leads to thiamine auxotrophy when fructose is used as a carbon source.

FIG. 5A is an image showing growth of cells in media using syngas as a carbon source in the absence “−” or presence “+” of thiamine. SB1::pKO31 denotes the cells with the integrated plasmid in the thiHSFG region.

FIG. 5B is a graph showing cell density (OD600) values for SB1 and SB1::pKO31 mutant grown in the absence of presence of thiamine. Taken together, FIGS. 5A and 5B show inactivation of SEQ ID NO: 1 (thiH), SEQ ID NO: 2 (thiS), SEQ ID NO: 3 (thiF), and SEQ ID NO: 4 (thiG) leads to thiamine auxotrophy when syngas is used as a carbon source.

FIG. 6A is a graph showing the growth of SB1::pKO31 cells in a bioreactor containing using syngas as a carbon source. Cells were cultured and allowed to reach steady state in the presence of thiamine. Thiamine was then removed from the media at 185 hr (black arrow) and compared to growth in a companion reactor in the presence of added thiamine.

FIG. 6B is a graph showing the ethanol productivity under identical conditions to FIG. 6A. Taken together, FIGS. 6A and 6B show inactivation of SEQ ID NO: 1 (thiH), SEQ ID NO: 2 (thiS), SEQ ID NO: 3 (thiF), and SEQ ID NO: 4 (thiG) leads to thiamine mixotrophy during stready state growth in bioreactors in syngas.

### DETAILED DESCRIPTION OF THE INVENTION

An aspect of the invention provides methods for producing from a gaseous substrate comprising CO, CO₂, and optionally H₂ at least one oxygenated product, the method comprising: (a) providing to a bioreactor: (1) a gaseous substrate comprising CO, CO₂, and optionally H₂, (2) at least one acetogenic carboxydotrophic bacterium, and (3) a liquid nutrient medium, and (b) providing conditions within the bioreactor for the at least one acetogenic carboxydotrophic bacterium to convert CO, CO₂, and optionally H₂ to at least one oxygenated product, wherein the at least one acetogenic carboxydotrophic bacterium comprises at least one gene selected from thiH, thiS, thiF, and thiG, and the conditions within the bioreactor create a bioreactor broth.

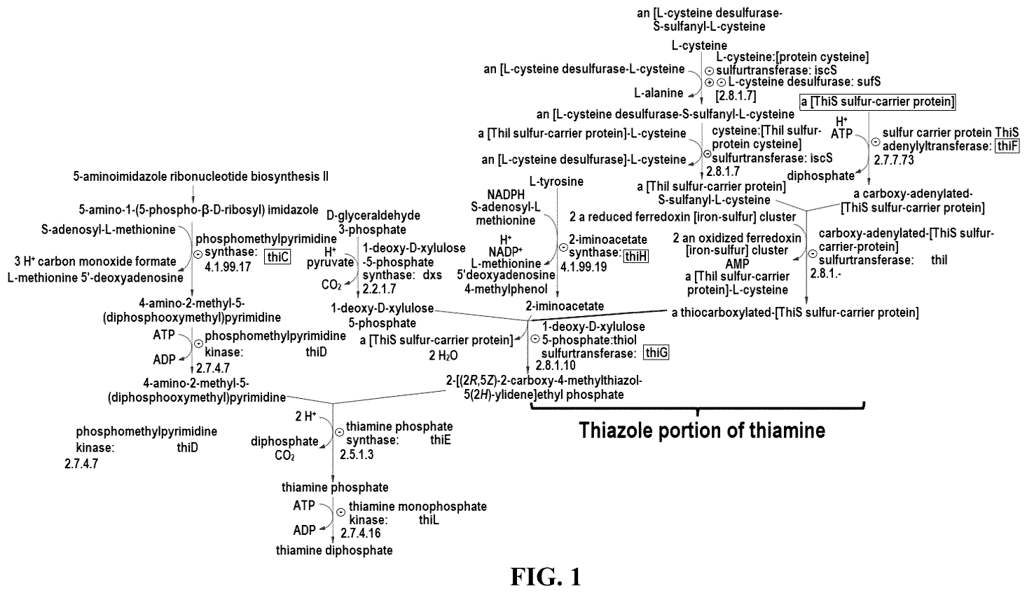

Thiamine may be produced in bacteria via an 11-step pathway (see FIG. 1). Bacteria that convert CO, CO₂, and optionally H₂ to at least one oxygenated product were previously known to require thiamine to survive and to produce oxygenated products. U.S. Pat. No. 10,415,043 discloses transforming Clostridium autoethanogenum with the thiamine biosynthesis protein (ThiC) (EC 4.1.99.17) derived from Clostridium ragsdalei. This transformation allows Clostridium autoethanogenum to grow without thiamine. The methods claimed herein are unexpected because in addition to thiC, Clostridria commonly lack thiG and thiH needed for synthesis of the thiazole component of thiamine (Annan et al., Applied Microbiology and Biotechnology, 103: 4633-4648 (2019)). Annan et al. suggested that these results indicate that “unknown genes or an unknown pathway exists that replaces thiG and ThiH” (Id. at 4646). However, it has been unexpectedly discovered that members of syngas utilizing Clostridia exist that contain the thiGSH pathway, and that if thiC is also present, these microbes do not require supplementation with exogeneous thiamine or thiazole-containing thiamine precursors for growth.

This unexpected discovery has several advantages. One advantage is that adding thiamine or thiazole-containing thiamine precursors to the fermentation can be costly and not adding thiamine or thiazole-containing thiamine precursors can therefore produce a cost savings. Additionally, some vitamins are sensitive to chemicals and temperature making the use of them undesirable due to process considerations and requiring design modifications. Also, the acidic nature of thiamine HCl, a common form may make the solutions of vitamins less stable depending on other components of the fermentation.

In an aspect, the at least one acetogenic carboxydotrophic bacterium comprises a sequence with at least about 60%, about 65%, about 70%, about 75%, about 80%, about 85%, about 86%, 87%, about 88%, about 89%, about 90%, about 91%, about 92%, about 93%, about 94%, about 95%, about 96%, about 97%, about 98%, about 99%, or about 100% identity to any one of SEQ ID NOs: 1-4.

Tyrosine lysase (thiH) is encoded by the thiH gene. In an aspect, the at least one acetogenic carboxydotrophic bacterium comprises a sequence with at least about 60%, about 65%, about 70%, about 75%, about 80%, about 85%, about 86%, 87%, about 88%, about 89%, about 90%, about 91%, about 92%, about 93%, about 94%, about 95%, about 96%, about 97%, about 98%, about 99%, or about 100% identity to thiH. In an aspect, the at least one acetogenic carboxydotrophic bacterium comprises a sequence with at least about 60%, about 65%, about 70%, about 75%, about 80%, about 85%, about 86%, 87%, about 88%, about 89%, about 90%, about 91%, about 92%, about 93%, about 94%, about 95%, about 96%, about 97%, about 98%, about 99%, or about 100% identity to the SEQ ID NO: 1

(ATGACCTTTTATGATGTAGTTCAAAGTCTTAAAGACTTTGATTATGACA
GCTTTTTTAAAAATATTTCAGACATGCAAATTCAACAAATAATTGCTAAA
GATAAATTGGACAAGTTTGATTTTTTAGCCCTACTTTCTCCTACTGCAGA
AAATCACATTGAAGAAATGGCTCAAAAGGCCCACAACATTTCCTTAAAAT
ATTTTGGCAAAAATGTACTTATATATACCCCAATGTACGTTGCCAATTAT
TGCTCAAATAGATGTGCCTACTGCGGATACAATTGTACAAATGATATAAA
CAGAAAAGTTCTATCTCTTGATGAAGTTGAAACAGAAGCTAAAGCTGTAC
ATGACAAGGGATTCAGACATGTAATTCTTTTAACGGGAGAATCTAGAAAA
TACTCTCCTGTTTCCTATATAAGGGATTGTGTTAAAATACTCAATAAATA
TTTCAGTTCAATATGCCTTGAAATCTATTCTCTTAAAAAAGAAGAATATG
AAGAACTTGTTAAAGCTGGTGCAGATAGTTTAACCATGTATCAGGAAACC
TACAATGAGGAAGTATATTCAAAGGTTCATCTTGGAGGTCCAAAGAAAAA
TTATAGATATAGACTTGAAACTCCAGAAAGAGCTTGTGAAGCTGGTATAT
ACTCCGTAGGTCTTGGTCCACTTTATGGTCTTTATGATTGGCGTGAGGAA
GCTTTTCTAGGTGGACTTCACGGTGCATACATCCAGAAAAAGTTTCCCGG
AGTAGATGTTAACTTTTCAGTTCCAAGAATACGCCCTCATGCAGGATCAT
TTAATAATCTTCACGAAATAAACGAGAAAACTGTAGTTCAAATACTACTG
GCACTTAAATTATTTATTCCAAGGTCAGGCACCAATATAACTACAAGAGA
ATGCTCAGAAATGAGAAATAACCTCATACCTCTTGGAGTCACTAAAATGT
CAGCTGGCGTATCTACAGCAGTTGGAGGCCACACTCAAGAAAATCCAGGT
GAAAAGCAATTTGATACATCTGACAAGAGAAGCCTTGAAGAAGTTAGAGA
AGCAATAACAAATAAGGGCTATTTTCCTGTAATGAAAGATTGGGAACCCA
TGATACTA).

ThiS carrier protein (thiS) is encoded by the thiS gene. In an aspect, the at least one acetogenic carboxydotrophic bacterium comprises a sequence with at least about 60%, about 65%, about 70%, about 75%, about 80%, about 85%, about 86%, 87%, about 88%, about 89%, about 90%, about 91%, about 92%, about 93%, about 94%, about 95%, about 96%, about 97%, about 98%, about 99%, or about 100% identity to thiS. In an aspect, the at least one acetogenic carboxydotrophic bacterium comprises a sequence with at least about 60%, about 65%, about 70%, about 75%, about 80%, about 85%, about 86%, 87%, about 88%, about 89%, about 90%, about 91%, about 92%, about 93%, about 94%, about 95%, about 96%, about 97%, about 98%, about 99%, or about 100% identity to SEQ ID NO: 2

(ATGATTGTAAATGGAGAAGAATTAAATTTTGAGAACGATATAACTGTAT
CTCAATTGCTGAACAAGTTAAATGTAAATGAAGAAACAGTTGTAGTTGAA
GTTGACTTAGAAATAGTAGACAGAGATGTCTATAAAACAAAAAAACTTTC
AAGCACTTCCAAAGTAGAAGTTATCCGTTATGTTGGAGGTGGC).

ThiS adenyltransferase (thiF) is encoded by the thiF gene. In an aspect, the at least one acetogenic carboxydotrophic bacterium comprises a sequence with at least about 60%, about 65%, about 70%, about 75%, about 80%, about 85%, about 86%, 87%, about 88%, about 89%, about 90%, about 91%, about 92%, about 93%, about 94%, about 95%, about 96%, about 97%, about 98%, about 99%, or about 100% identity to thiF. In an aspect, the at least one acetogenic carboxydotrophic bacterium comprises a sequence with at least about 60%, about 65%, about 70%, about 75%, about 80%, about 85%, about 86%, 87%, about 88%, about 89%, about 90%, about 91%, about 92%, about 93%, about 94%, about 95%, about 96%, about 97%, about 98%, about 99%, or about 100% identity to SEQ ID NO: 3. In an aspect, the at least one acetogenic carboxydotrophic bacterium does not comprise SEQ ID NO: 3

(ATGCCATTGACAGATAAAGAGCGTGAAAAGTATGCAAGACACCTAGTAT
TAAAAGAAATTGGTGCGGCAGGTCAGGAAAAACTTTTAAGCTCCAGGATT
TTAATTATAGGCACTGGAGGCCTGGGTTCCCCAGCCTCTGCATATCTTGC
TGCAGCAGGTATCGGGACACTAGGACTTGTAGACTTTGATAAAGTAGAAC
TGTCAAACCTTCAAAGACAAATAATACACATGACAAAAAATATAGGGAAA
CCAAAACTTTTATCTGCAAAAGAAACTTTAAACAATATAAATCCTGATAT
GAATGTAATAACTTATAATGAACATCTTGACCACAATAACACAGCTGACA
TAATAAATGACATGAATTATGATTTCATTTTGGATTGTACAGATAACTTT
GAGTCAAAATTTCTTATAAACGATGCTTGTGTTGCCTTAAAAAAACCTTT
TTCACATGGAGGTGTTATAAGATTTAGAGGACAAACCATGACTTATATTC
CCGGTAAAGGTCCATGTTATAGGTGTGTTTTCGTAAATCCACCCCTGGAT
GGCGTTGTTCCCACTTCAAAGCAAGTTGGAATAATTGGCACTTCACCAGG
AGTTATAGGCACCATTCAGGCTATGGAGGCCATCAAATATATTCTTCATA
TAGGAAATCTCCTTACAGAACACCTTTTAATATACGATGGACTAAAAATG
GATTTCAGAAAAATCAAAATATCCAAGAGAAACGATTGTGCAGCCTGTGG
TAAAAAACATCTA).

Thiazole synthase (thiG) is encoded by the thiG gene. In an aspect, the at least one acetogenic carboxydotrophic bacterium comprises a sequence with at least about 60%, about 65%, about 70%, about 75%, about 80%, about 85%, about 86%, 87%, about 88%, about 89%, about 90%, about 91%, about 92%, about 93%, about 94%, about 95%, about 96%, about 97%, about 98%, about 99%, or about 100% identity to thiG. In an aspect, at least one acetogenic carboxydotrophic bacterium comprises a sequence with at least about 60%, about 65%, about 70%, about 75%, about 80%, about 85%, about 86%, 87%, about 88%, about 89%, about 90%, about 91%, about 92%, about 93%, about 94%, about 95%, about 96%, about 97%, about 98%, about 99%, or about 100% identity to SEQ ID NO: 4

(ATGGATGAACTTGTAATAGCAAATAAAAAAATAAAAAGCAGATTTTTCA
TTGGAACAGGGAAATTTTCGTCAAATGAAATAGTTCCAGATATTATTAAA
AGCTCTGAAGCACAGGTTATAACAGTAGCCTTAAGACGTATCGACATACA
TGCTACAGATTCAAGAGATAATATACTGAATTTTATAGATAAAGATTGCA
TACTTGTGGCAAATACATCCGGTGCAAGAAATGCAGAGGAAGCTATACGA
CTTGCTCATTTAGCTAAAGCAACAGGATGTGGAAATTGGGTTAAAATTGA
GGTTATTTCAGATAGCAAATATCTTCTTCCTGATAACTATGAAACTTTAA
AAGCTACTGAAGTACTTGTAAAAGAAGGTTTTATAGCACTTCCATATATA
AGTCCAAATTTAATGGACGCAAAAAGACTTGTAGACGCAGGTGCCTCTGC
AGTCATGCCCTTAGGTTCTCCTATAGGTACAAATAGAGGTCTTAAAACCA
GGGAACTTTTAAAAATTCTAATTGATGAAATAGACATTCCTGTAGTTGTA
GATGCAGGTATAGGAAAACCCTCTCACGCCGCAGAAGCCATGGAAATGGG
TGCAGATGCAGTACTTGCAAATACTGCTCTTGCAACAGCAAAAGATCCAG
TACTCATAGCTGAAGCTTTCAAACTTGCAGTACAAGCAGGAAGAAAAGCC
TTTCTTTCAAAACCTGGTATTGAAAGAGAATTGGCCAGTGCTTCATCACC
ACTCACAGGATTTTTAAGA).

In an aspect, the at least one acetogenic carboxydotrophic bacterium comprises thiH and thiS. In an aspect, the at least one acetogenic carboxydotrophic bacterium comprises thiH and thiF. In an aspect, the at least one acetogenic carboxydotrophic bacterium comprises thiH and thiG. In an aspect, the at least one acetogenic carboxydotrophic bacterium comprises thiS and thiF. In an aspect, the at least one acetogenic carboxydotrophic bacterium comprises thiS and thiG. In an aspect, the at least one acetogenic carboxydotrophic bacterium comprises thiF and thiG.

In an aspect, the at least one acetogenic carboxydotrophic bacterium comprises thiH, thiS, and thiF. In an aspect, the at least one acetogenic carboxydotrophic bacterium comprises thiS, thiF, and thiG. In an aspect, the at least one acetogenic carboxydotrophic bacterium comprises thiH, thiS, and thiG. In an aspect, the at least one acetogenic carboxydotrophic bacterium comprises thiH, thiF, and thiG. In a further aspect, the at least one acetogenic carboxydotrophic bacterium comprises thiH, thiS, thiF, and thiG.

In an aspect, thiH, thiS, thiF, or thiG is an endogenous gene. In an aspect, the at least one acetogenic carboxydotrophic bacterium is engineered (e.g., by adding an appropriate promoter) to have a greater expression of endogenous thiH, thiS, thiF, and/or thiG, than a non-engineered acetogenic carboxydotrophic bacterium.

In an aspect, the at least one acetogenic carboxydotrophic bacterium is recombinant. In an aspect, the at least one acetogenic carboxydotrophic bacterium was engineered to include at least one gene selected from thiH, thiS, thiF, and thiG. In an aspect, thiH, thiS, thiF, or thiG is an exogenous gene. An “exogenous gene” is a gene which originates outside of the acetogenic carboxydotrophic bacterium to which the gene is introduced. Exogenous genes may be derived from any appropriate source, including, but not limited to, the acetogenic carboxydotrophic bacterium to which they are to be introduced (for example, a parental acetogenic carboxydotrophic bacterium from which the recombinant acetogenic carboxydotrophic bacterium is derived), strains or species of acetogenic carboxydotrophic bacterium which differ from the acetogenic carboxydotrophic bacterium to which they are to be introduced, or they may be artificially or recombinantly created. In one aspect, the exogenous gene is introduced to increase expression of or over-express a particular gene (for example, by increasing the copy number of the gene), or introducing a strong or constitutive promoter to increase expression). In another aspect, the exogenous gene represents a gene not naturally present within the acetogenic carboxydotrophic bacterium to which the gene is to be introduced and allows for the expression of a product not naturally present within the acetogenic carboxydotrophic bacterium or increased expression of a gene native to the acetogenic carboxydotrophic bacterium (for example, in the case of introduction of a regulatory element such as a promoter). The exogenous gene may be adapted to integrate into the genome of the acetogenic carboxydotrophic bacterium to which the exogenous gene is to be introduced.

In an aspect, the at least one acetogenic carboxydotrophic bacterium is non-naturally occurring. “Non-naturally occurring” as used herein refers to an acetogenic carboxydotrophic bacterium that has been modified by the hand of man and has at least one genetic modification not found in a naturally occurring strain of the referenced species, i.e., not found in the wild-type strain of the referenced species.

In an aspect, the at least one acetogenic carboxydotrophic bacterium is a Clostridium bacterium.

An aspect of the invention provides methods for producing from a gaseous substrate comprising CO, CO₂, and optionally H₂ at least one oxygenated product, the method comprising: (a) providing to a bioreactor: (1) a gaseous substrate comprising CO, CO₂, and optionally H₂, (2) at least one acetogenic carboxydotrophic bacterium, and (3) a liquid nutrient medium, and (b) providing conditions within the bioreactor for the at least one acetogenic carboxydotrophic bacterium to convert the gaseous substrate to at least one oxygenated product, wherein the at least one acetogenic carboxydotrophic bacterium does not require thiamine or thiazole-containing thiamine precursors, and the conditions within the bioreactor create a bioreactor broth.

As used herein, “thiamine or thiazole-containing thiamine precursors” includes compounds that contain a thiazole moiety.

As used herein, “thiazole-containing thiamine precursors” refers to thiazole compounds that are precursors in the biosynthesis of thiamine.

An aspect of the invention provides methods for operating a syngas fermentation without the addition of thiamine or thiazole-containing thiamine precursors, the method comprising: (a) providing to a bioreactor: (1) a gaseous substrate comprising CO, CO₂, and optionally H₂, (2) at least one acetogenic carboxydotrophic bacterium, and (3) a liquid nutrient medium that does not contain thiamine or thiazole-containing thiamine precursors, and (b) providing conditions within the bioreactor for the at least one acetogenic carboxydotrophic bacterium to convert CO, CO₂, and optionally H₂ to at least one oxygenated product, wherein the conditions within the bioreactor create a bioreactor broth, and thiamine or thiazole-containing thiamine precursors is not added to (i) the liquid nutrient medium or (ii) the bioreactor broth before or during syngas fermentation.

As used herein, thiamine includes salts thereof, including thiamine HCl, thiamine mononitrate, and thiamine nitrate.

An aspect of the invention provides methods for producing from a gaseous substrate comprising CO, CO₂, and optionally H₂ at least one oxygenated product, the method comprising: (a) providing to a bioreactor: (1) a gaseous substrate comprising CO, CO₂, and optionally H₂, (2) at least one acetogenic carboxydotrophic bacterium, and (3) a liquid nutrient medium, and (b) providing conditions within the bioreactor for the at least one acetogenic carboxydotrophic bacterium to convert the gaseous substrate to at least one oxygenated product, wherein the at least one acetogenic carboxydotrophic bacterium comprises at least one gene (e.g., exogenous gene) selected from thiH, thiS, thiF, and thiG.

An aspect of the invention provides methods for using a broth deficient in thiamine or thiazole-containing thiamine precursors as a screening tool for suitable ethanol producing acetogenic carboxydotrophic bacteria strains, the method comprising: (a) providing to a bioreactor in the absence of thiamine or thiazole-containing thiamine precursors: (1) a gaseous substrate comprising CO, CO₂, and optionally H₂, (2) at least one acetogenic carboxydotrophic bacterium, and (3) a liquid nutrient medium, and (b) providing conditions within the bioreactor for the at least one acetogenic carboxydotrophic bacterium to convert the gaseous substrate to at least one oxygenated product, wherein the conversion of CO, CO₂, and optionally H₂ to at least one oxygenated product indicates the presence of a suitable ethanol producing acetogenic carboxydotrophic bacteria strain.

An aspect of the invention provides methods for controlling bacterial contamination in a bioreactor, the method comprising: (a) providing to a bioreactor in the absence of thiamine or thiazole-containing thiamine precursors: (1) a gaseous substrate comprising CO, CO₂, and optionally H₂, (2) at least one acetogenic carboxydotrophic bacterium, and (3) a liquid nutrient medium, and (b) providing conditions within the bioreactor for the at least one acetogenic carboxydotrophic bacterium to convert the gaseous substrate to at least one oxygenated product, wherein the absence of exogenously supplied thiamine or thiazole-containing thiamine precursors in the bioreactor prevents contamination of the bioreactor broth with non-desirable acetogenic carboxydotrophic bacteria.

As used herein, “does not contain thiamine or thiazole-containing thiamine precursors” and “absence of thiamine or thiazole-containing thiamine precursors” means that less there is less than 0.05 mg of thiamine or thiazole-containing thiamine precursors per 100 L of fermentation broth.

As used herein, “exogenously supplied thiamine or thiazole-containing thiamine precursors” means that thiamine or thiazole-containing thiamine precursors are not added to the fermentation broth, bioreactor, or liquid nutrient medium.

In an aspect, no more than 0.05 mg of thiamine or thiazole-containing thiamine precursors is added per 100 L of bioreactor or fermentation broth. In this regard, no more than about 0.05 mg, about 0.06 mg, about 0.07 mg, about 0.08 mg, about 0.09 mg, about 0.1 mg, about 0.2 mg, about 0.3 mg, or about 0.4 mg of thiamine is added per 100 L of bioreactor or fermentation broth.

In an aspect, no more than 0.05 mg of a thiamine or thiazole-containing thiamine precursors is per 100 L is added to the bioreactor or fermentation broth. In this regard, no more than about 0.05 mg, about 0.06 mg, about 0.07 mg, about 0.08 mg, about 0.09 mg, about 0.1 mg, about 0.2 mg, about 0.3 mg, or about 0.4 mg of a thiazole containing compound per 100 L is added to the bioreactor or fermentation broth.

In an aspect, the oxygenated product is acetic acid (“acetic acid” and “acetate” are used interchangeably herein), butyrate, butanol, propionate, and propanol. In an aspect, the oxygenated product is ethanol.

In an aspect, the gaseous substrate comprising CO, CO₂, and optionally H₂ is a synthesis gas (syngas), such as syngas obtained by gasification of coal or refinery residues, gasification of biomass or lignocellulosic material, or reforming of natural gas. In another aspect, the syngas may be obtained from the gasification of municipal solid waste or industrial solid waste.

The gaseous substrate may comprise at least CO, such as about 1, about 2, about 5, about 10, about 20, about 30, about 40, about 50, about 60, about 70, about 80, about 90, or about 100 mol % CO. The gaseous substrate may comprise a range of CO, such as about 20 to about 80, about 30 to about 70, or about 40 to about 60 mol % CO. Preferably, the substrate comprises about 40 to about 70 mol % CO (e.g., steel mill or blast furnace gas), about 20 to about 30 mol % CO (e.g., basic oxygen furnace gas), or about 15 to about 45 mol % CO (e.g., syngas). In some aspects, the gaseous substrate may comprise a relatively low amount of CO, such as about 1 to about 10 or about 1 to about 20 mol % CO. In some aspects, the gaseous substrate comprises no or substantially no (<about 1 mol %) CO.

The gaseous substrate may optionally comprise H₂. For example, the gaseous substrate may comprise about 1, about 2, about 5, about 10, about 15, about 20, or about 30 mol % H₂. In some aspects, the gaseous substrate may comprise a relatively high amount of H₂, such as about 60, about 70, about 80, or about 90 mol % H₂. In another aspect, the gaseous substrate comprises no or substantially no (<about 1 mol %) H₂ (e.g., when derived from steel mill gas). The H₂ may be derived from or produced by any suitable process, including the formation of H₂ using electrodes.

The gaseous substrate may comprise CO₂. For example, the gaseous substrate may comprise from about 1 to about 80, from about 1 to about 70, from about 1 to about 60, from about 1 to about 50, from about 1 to about 40, from about 1 to about 30, from about 1 to about 25, from about 1 to about 20, from about 1 to about 15, from about 1 to about 10, from about 1 to about 5, from about 1 to about 4, from about 1 to about 3, or from about 2 to about 3 mol % CO₂. In some aspects, the gaseous substrate may comprise less than about 20, about 15, about 10, or about 5 mol % CO₂. In another aspect, the gaseous substrate comprises no or substantially no (<about 1 mol %) CO₂.

In an aspect, the at least one acetogenic carboxydotrophic bacterium is cultured in the bioreactor to produce an acetogenic carboxydotrophic bacteria culture. In an aspect, the acetogenic carboxydotrophic bacteria culture continuously produces at least one oxygenated product for more than about 24 hours, about 36 hours, about 48 hours, about 72 hours, about 96 hours, about 120 hours, about 144 hours, about 168 hours, about 192 hours, about 216 hours, about 250 hours, about 300 hours, about 400 hours, about 500 hours, about 600 hours, about 700 hours, about 800 hours, about 900 hours, about 1,000, 1,100 hours, about 1,200 hours, about 1,300 hours, about 1,400 hours, about 1,500 hours, about 1,600 hours, about 1,700 hours, about 1,800 hours, about 1,900 hours, about 2,000 hours, about 2,500 hours, or about 3,000 hours.

In an aspect, the acetogenic carboxydotrophic bacteria culture continuously produces at least about 0.1 g/L/day of the at least one oxygenated product (e.g., at least about 0.1 g/L/day, at least about 0.2 g/L/day, at least about 0.3 g/L/day, at least about 0.4 g/L/day, at least about 0.5 g/L/day, at least about 0.6 g/L/day, at least about 0.7 g/L/day, at least about 0.8 g/L/day, at least about 0.9 g/L/day, at least about 1 g/L/day, at least about 1.1 g/L/day, at least about 1.2 g/L/day, at least about 1.3 g/L/day, at least about 1.4 g/L/day, at least about 1.5 g/L/day, at least about 1.6 g/L/day, at least about 1.7 g/L/day, at least about 1.8 g/L/day, at least about 1.9 g/L/day, or at least about 2.0 g/L/day).

In an aspect, the inventive methods further comprise wherein the conditions within the bioreactor create a bioreactor broth.

In an aspect, the inventive methods further comprise removing the bioreactor broth from the bioreactor to produce a removed broth.

In an aspect, the inventive methods further comprise removing the at least one oxygenated product from the removed broth to produce an oxygenated product-depleted removed broth. The oxygenated products may be separated or purified from the fermentation broth using any method or combination of methods known in the art, including, for example, fractional distillation, evaporation, pervaporation, gas stripping, phase separation, and extractive fermentation, including for example, liquid-liquid extraction. In certain aspects, target products are recovered from the fermentation broth by continuously removing a portion of the broth from the bioreactor, recovering one or more oxygenated products from the fermentation broth, and separating cells (and components thereof) from the broth (e.g., filtration). Alcohols may be recovered, for example, by distillation.

In an aspect, the inventive methods further comprise removing cells (and components thereof) of the culture from the removed broth and/or the oxygenated product-depleted removed broth. Removal of the cells (and components thereof) can be carried out in any suitable means, for example, by cyclones, filtration, or centrifugation. In an aspect, the majority (i.e., greater than 50%) of the cells (and components thereof) of the non-naturally occurring acetogenic carboxydotrophic bacteria culture are removed. In an aspect, the removed cells (and components thereof) of the acetogenic carboxydotrophic bacteria culture are removed from the removed broth before one or more oxygenated products are recovered from the fermentation broth. In an aspect, the removed cells (and components thereof) of the acetogenic carboxydotrophic bacteria culture are removed from the removed broth after one or more oxygenated products are recovered from the fermentation broth.

In an aspect, the inventive methods further comprise providing the oxygenated product-depleted removed broth to the bioreactor. Additional nutrients (e.g., non-thiamine B vitamins and metals) may be added to the removed broth to replenish the oxygenated product-depleted removed broth before the oxygenated product-depleted removed broth is returned to the bioreactor.

In an aspect, at least about 1 gram, at least about 2 grams, at least about 3 grams, at least about 4 grams, at least about 5 grams, at least about 6 grams, at least about 7 grams, at least about 8 grams, at least about 9 grams, at least about 10 grams, at least about 11 grams, at least about 12 grams, at least about 13 grams, at least about 14 grams, at least about 15 grams, or at least about 20 grams, or at least about 25 grams, or at least about 30 grams, or at least about 35 grams, or at least about 40 grams, or at least about 45 grams, or at least about 50 grams, or at least about 55 grams, or at least about 60 grams, or at least about 65 grams, or at least about 70 grams, or at least about 75 grams, or at least about 80 grams, or at least about 85 grams, or at least about 90 grams, or at least about 95 grams, or at least about 100 grams, or from about 1 to about 100 grams, or from about 16 to about 20 grams of the at least one oxygenated product are produced per liter of removed broth.

In an aspect, more than about 30%, about 35%, about 40%, about 45%, about 50%, about 55%, about 60%, about 65%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the oxygenated product-depleted removed broth is provided back to the bioreactor. In this regard, from about 1% to about 100% of the oxygenated product-depleted removed broth is provided back to the bioreactor, for example, from about 1% to about 30%, from about 1% to about 35%, from about 1% to about 40%, from about 1% to about 45%, from about 1% to about 50%, from about 1% to about 55%, from about 1% to about 60%, from about 1% to about 65%, from about 1% to about 70%, from about 1% to about 75%, or from about 1% to about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the removed broth is provided back to the bioreactor. In an alternative aspect, from about 50% to about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the oxygenated product-depleted removed broth is provided back to the bioreactor.

In an aspect, the fermentation is a co-culture and includes at least one acetogenic carboxydotrophic bacterium and an additional bacterium. In an aspect, the additional bacterium is an acetogenic carboxydotrophic bacterium. In another aspect, the additional bacterium is not an acetogenic carboxydotrophic bacterium. In an aspect, the additional bacterium is a butyrate producing bacterium (see e.g., U.S. Pat. No. 9,469,860 B2, incorporated herein by reference). In an aspect, the additional bacterium is a propionate producing bacterium (see e.g., US Patent Application 2014/0273123 A1, incorporated herein by reference).

Typically, the fermentation is performed in a bioreactor. The term “bioreactor” includes a culture/fermentation device consisting of one or more vessels, towers, or piping arrangements, such as a continuous stirred tank reactor (CSTR), immobilized cell reactor (ICR), trickle bed reactor (TBR), bubble column, gas lift fermenter, static mixer, or other vessel or other device suitable for gas-liquid contact. In certain aspects, the bioreactor may comprise a first growth reactor and a second culture/fermentation reactor. The substrate may be provided to one or both of these reactors. As used herein, the terms “culture” and “fermentation” are used interchangeably. These terms encompass both the growth phase and product biosynthesis phase of the culture/fermentation process.

As used herein, a “bioreactor” refers to a bioreactor assembly. A bioreactor assembly is a group of one or more vessels suitable to contain aqueous broth and acetogenic carboxydotrophic bacteria for the bioconversion. The bioreactor assembly may contain associated equipment such as injectors, recycle loops, and agitators.

Any suitable bioreactor assembly may be used in the methods of the invention. The bioreactor assembly used in the methods of the invention can be the bioreactor assembly used for the bioconversion of syngas, or the bioreactor assembly used in the methods of the invention can be separate from the bioreactor assembly used for the bioconversion of syngas. The bioreactor assemblies for use in the methods of the invention include, but are not limited to, column reactors; bubble columns; jet loop reactors; stirred tank reactors; fluidized bed reactors; trickle bed reactors; biofilm reactors, including, but not limited to membrane bioreactors; and static mixer reactors including, but not limited to, pipe reactors. One or more bioreactors may be used, and when two or more bioreactors are used, they may be in parallel or sequential operation. The bioreactor assembly can, but is not always required to, include heat exchangers; solids separation unit operations such as centrifuges, settling ponds, and filters; gas/liquid separation unit operations; pumps; and equipment useful for monitoring and control of the bioreactor assembly.

A separate bioreactor assembly can, if desired, be integrated into the facility for the bioconversion of syngas to oxygenated organic compound. For instance, where the facility contains a distillation assembly, the distillation assembly may be used to remove at least a portion of the oxygenated product and to denature the aqueous fermentation broth.

As used herein, “biomass” refers to living or previously (e.g., recently) living biological material, including plants and animals, and contains at least hydrogen, oxygen, and carbon. Biomass typically also contains nitrogen, phosphorus, sulfur, sodium, potassium, and trace metals. The chemical composition of biomass can vary from source to source and even within a source. Sources of biomass include, but are not limited to, harvested plants such as wood, grass clippings, and yard waste, switchgrass, corn (including corn stover), hemp, sorghum, sugarcane (including bagas), and the like, and waste such as garbage and municipal solid waste. Biomass does not include fossil fuels such as coal, natural gas, and petroleum.

Fossil carbonaceous materials, or fossil fuels, include, but are not limited to, natural gas; petroleum including carbonaceous streams from the refining or other processing of petroleum including, but not limited to, petroleum coke, lignite, and coal.

As used herein, “aqueous broth,” or “aqueous fermentation broth,” refers to a liquid water phase which may contain dissolved compounds including, but not limited to hydrogen, carbon monoxide, and carbon dioxide.

Intermittently means from time to time and may be at regular or irregular time intervals.

Syngas means a gas, regardless of source, containing at least one of hydrogen and carbon monoxide and may, and usually does, contain carbon dioxide.

Syngas can be made from many carbonaceous feedstocks. These include sources of hydrocarbons such as natural gas, biogas, biomass, especially woody biomass, gas generated by reforming hydrocarbon-containing materials, peat, petroleum coke, coal, waste material such as debris from construction and demolition, municipal solid waste, and landfill gas. Syngas is typically produced by a gasifier or reformer (steam, autothermal, or partial oxidation). Any of the aforementioned biomass sources are suitable for producing syngas. The syngas produced thereby will typically contain from about 10 to about 60 mole % CO, at least about 1 mole % CO₂, and preferably between about 35 and about 65 mole % H₂. The syngas may also contain N₂ and CH₄ as well as trace components such as H₂S, COS, NH₃, and HCN. Other sources of the gas substrate include gases generated during petroleum and petrochemical processing and from industrial processes. These gases may have substantially different compositions than typical syngas and may be essentially pure hydrogen or essentially pure carbon monoxide. The gas substrate may be obtained directly from gasification or from petroleum and petrochemical processing or industrial processes or may be obtained by blending two or more streams. Also, the gas substrate may be treated to remove or alter the composition including, but not limited to, removing components by chemical or physical sorption, membrane separation, and selective reaction.

The product oxygenated organic compounds produced in the methods of this invention will depend upon the acetogenic carboxydotrophic bacterium or combination of acetogenic carboxydotrophic bacteria used for the fermentation and the conditions of the fermentation.

The aqueous broth is maintained under anaerobic fermentation conditions including a suitable temperature, for example, between about 25° C. and about 60° C., or in the range of about 30° C. to about 40° C. The conditions of fermentation, including the density of acetogenic carboxydotrophic bacterium and aqueous fermentation broth composition are preferably sufficient to achieve the sought conversion efficiency of hydrogen and carbon monoxide. The pH of the aqueous broth is acidic, for example, between about 4 and about 6.5.

The rate of supply of the syngas under steady state conditions to a bioreactor is preferably such that the rate of transfer of carbon monoxide and hydrogen to the liquid phase matches the rate that carbon monoxide and hydrogen are bioconverted. The rate at which carbon monoxide and hydrogen can be consumed will be affected by the nature of the acetogenic carboxydotrophic bacteria, the concentration of the acetogenic carboxydotrophic bacteria in the aqueous fermentation broth and the fermentation conditions. As the rate of transfer of carbon monoxide and hydrogen to the aqueous fermentation broth is a parameter for operation, conditions affecting the rate of transfer such as interfacial surface area between the gas and liquid phases and driving forces are important. Preferably the feed gas is introduced into the bioreactor in the form of microbubbles. Often the microbubbles have diameters in the range of about 0.01 to about 0.5 millimeter, or about 0.02 to about 0.3 millimeter.

In another aspect, the disclosure provides a method of preparing animal feed. As used herein, animal feed can be any suitable type of animal feed, such as, for example, aquatic culture (fish feed), poultry feed, cattle feed, hog feed, bird feed, etc. In an aspect, the at least one acetogenic carboxydotrophic bacterium and/or the acetogenic carboxydotrophic bacteria culture are effective for use as animal feed. In a further aspect, the disclosure provides methods of preparing animal feed, the methods comprising: providing to a bioreactor: (1) a gaseous substrate comprising CO, CO₂, and optionally H₂, (2) at least one acetogenic carboxydotrophic bacterium comprising at least one gene selected from thiH, thiS, thiF, and thiG, and (3) a liquid nutrient medium, providing conditions within the bioreactor for the at least one acetogenic carboxydotrophic bacterium to convert the gaseous substrate to at least one oxygenated product, and removing the at least one acetogenic carboxydotrophic bacterium from the bioreactor, wherein the at least one acetogenic carboxydotrophic bacterium is effective for use as animal feed. In another aspect, the disclosure provides methods of preparing animal feed, the methods comprising: providing to a bioreactor in the absence of thiamine or thiazole-containing thiamine precursors: (1) a gaseous substrate comprising CO, CO₂, and optionally H₂, (2) at least one acetogenic carboxydotrophic bacterium, and (3) a liquid nutrient medium, providing conditions within the bioreactor for the at least one acetogenic carboxydotrophic bacterium to convert the gaseous substrate to at least one oxygenated product, and removing the at least one acetogenic carboxydotrophic bacterium from the bioreactor, wherein the at least one acetogenic carboxydotrophic bacterium is effective for use as animal feed.

In some embodiments, the method of preparing animal feed is useful for producing aquatic culture containing relatively low amounts of one or more toxic metals, such as mercury, iron, nickel, etc.

In another aspect, the disclosure provides a method of preparing fertilizer. In an aspect, the at least one acetogenic carboxydotrophic bacterium and/or the acetogenic carboxydotrophic bacteria culture are effective for use as fertilizer. In a further aspect, the disclosure provides methods of preparing fertilizer, the methods comprising: providing to a bioreactor: (1) a gaseous substrate comprising CO, CO₂, and optionally H₂, (2) at least one acetogenic carboxydotrophic bacterium comprising at least one gene selected from thiH, thiS, thiF, and thiG, and (3) a liquid nutrient medium, (b) providing conditions within the bioreactor for the at least one acetogenic carboxydotrophic bacterium to convert the gaseous substrate to at least one oxygenated product, and (c) removing the at least one acetogenic carboxydotrophic bacterium from the bioreactor, wherein the at least one acetogenic carboxydotrophic bacterium is effective for use as fertilizer. In another aspect, the disclosure provides methods of preparing fertilizer, the methods comprising: providing to a bioreactor in the absence of thiamine or thiazole-containing thiamine precursors: (1) a gaseous substrate comprising CO, CO₂, and optionally H₂, (2) at least one acetogenic carboxydotrophic bacterium, and (3) a liquid nutrient medium, providing conditions within the bioreactor for the at least one acetogenic carboxydotrophic bacterium to convert the gaseous substrate to at least one oxygenated product, and removing the at least one acetogenic carboxydotrophic bacterium from the bioreactor, wherein the at least one acetogenic carboxydotrophic bacterium is effective for use as fertilizer.

In an aspect, the at least one acetogenic carboxydotrophic bacterium and/or the acetogenic carboxydotrophic bacteria culture are effective for landfill application or land application as fertilizer or animal feed. In an aspect, the at least one acetogenic carboxydotrophic bacterium and/or the acetogenic carboxydotrophic bacteria culture are useful for fertilizer, animal feed, landfill application, and/or land application contain protein, fat, carbohydrate, and/or minerals, e.g., 86% protein, 2% fat, 2% minerals, 10% carbohydrate. The at least one acetogenic carboxydotrophic bacterium and/or the acetogenic carboxydotrophic bacteria culture can be effective for landfill application (e.g., to Class A solids), land application as fertilizer, or feed for animals, such as aquatic culture (fish feed), poultry feed, cattle feed, hog feed, etc. In the case of fish feed, advantageously, in some embodiments, the fish feed contains less total toxic metals as compared with conventional fish meal.

In some aspects, the at least one acetogenic carboxydotrophic bacterium and/or the acetogenic carboxydotrophic bacteria culture contain about 25 to about 50 wt. % solids, such as about 25 to about 40 wt. % solids. The amount of recovered solids is advantageous because it contains protein, carbohydrates, minerals, and potentially vitamins of nutritional value for plants and animals.

The at least one acetogenic carboxydotrophic bacterium and/or the acetogenic carboxydotrophic bacteria culture can be used wet or dry. For example, in some aspects, the at least one acetogenic carboxydotrophic bacterium and/or the acetogenic carboxydotrophic bacteria culture are effective for use as a wet fertilizer. In some aspects, the method further comprises drying the at least one acetogenic carboxydotrophic bacterium and/or the acetogenic carboxydotrophic bacteria culture forms a “dried cake”, and the resulting dried cake is effective for use as dry fertilizer, animal feed or fish feed, or any combination thereof. The drying of the at least one acetogenic carboxydotrophic bacterium and/or the acetogenic carboxydotrophic bacteria culture can also be used as a means to concentrate the at least one acetogenic carboxydotrophic bacterium and/or the acetogenic carboxydotrophic bacteria culture. However, the removed at least one acetogenic carboxydotrophic bacterium and/or the acetogenic carboxydotrophic bacteria culture can be dried using any suitable means.

The respective compositions of the animal feed and fertilizer are generally similar because they are mainly composed of microbial proteins and/or carbohydrates. In some embodiments, the animal feed and/or fertilizer contains protein (e.g. from about 30 wt. % to about 90 wt. %, such as from about 60 wt. % to about 90 wt. %), fat (e.g. from about 1 wt. % to about 12 wt. %, such as from about 1 wt. % to about 3 wt. %), carbohydrate (e.g. from about 5 wt. % to about 60 wt. %, such as from about 15 wt. % to about 60 wt. %, or from about 5 wt. % to about 15 wt. %) and/or minerals such as sodium, potassium, copper etc. (e.g. from about 1 wt. % to about 20 wt. %, such as from about 1 wt. % to about 3 wt. %). For example, the animal feed and/or fertilizer can contain about 86% protein, about 2% fat, about 2% minerals, and about 10% carbohydrate. Aspects, including embodiments, of the subject matter described herein may be beneficial alone or in combination, with one or more other aspects or embodiments. Without limiting the foregoing description, certain non-limiting aspects of the disclosure numbered 1-24 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

(1) A method for producing from a gaseous substrate comprising CO, CO₂, and optionally H₂ at least one oxygenated product, the method comprising:
providing to a bioreactor: (1) a gaseous substrate comprising CO, CO₂, and optionally H₂, (2) at least one acetogenic carboxydotrophic bacterium, and (3) a liquid nutrient medium, and
providing conditions within the bioreactor for the at least one acetogenic carboxydotrophic bacterium to convert the gaseous substrate to at least one oxygenated product, wherein the at least one acetogenic carboxydotrophic bacterium comprises at least one gene selected from thiH, thiS, thiF, and thiG.

(2) The method of aspect 1, wherein the at least one acetogenic carboxydotrophic bacterium comprises a sequence with at least 90% identity to SEQ ID NO: 1 (thiH).

(3) The method of aspect 1, wherein the at least one acetogenic carboxydotrophic bacterium comprises a sequence with at least 90% identity to SEQ ID NO: 2 (thiS).

(4) The method of aspect 1, wherein the at least one acetogenic carboxydotrophic bacterium comprises a sequence with at least 90% identity to SEQ ID NO: 3 (thiF).

(5) The method of aspect 1, wherein the at least one acetogenic carboxydotrophic bacterium comprises a sequence with at least 90% identity to SEQ ID NO: 4 (thiG).

(6) The method of aspect 1, wherein the at least one acetogenic carboxydotrophic bacterium comprises thiH, thiS, thiF, and thiG.

(7) A method for producing from a gaseous substrate comprising CO, CO₂, and optionally H₂ at least one oxygenated product, the method comprising:
providing to a bioreactor: (1) a gaseous substrate comprising CO, CO₂, and optionally H₂, (2) at least one acetogenic carboxydotrophic bacterium, and (3) a liquid nutrient medium, and
providing conditions within the bioreactor for the at least one acetogenic carboxydotrophic bacterium to convert the gaseous substrate to at least one oxygenated product,
wherein the at least one acetogenic carboxydotrophic bacterium does not require exogenously supplied thiazole-containing compounds.

(8) The method of any one of aspects 1-7, wherein no more than 0.05 mg of thiamine or thiazole-containing thiamine precursors per 100 L is provided to the bioreactor.

(9) A method of operating a syngas fermentation without the addition of thiamine or thiazole-containing thiamine precursors, the method comprising:
providing to a bioreactor: (1) a gaseous substrate comprising CO, CO₂, and optionally H₂, (2) at least one acetogenic carboxydotrophic bacterium, and (3) a liquid nutrient medium that does not contain thiamine or a thiazole-containing thiamine precursors, and
providing conditions within the bioreactor for the at least one acetogenic carboxydotrophic bacterium to convert the gaseous substrate to at least one oxygenated product,
wherein the conditions within the bioreactor create a bioreactor broth, and thiamine or thiazole-containing thiamine precursors are not added to (i) the liquid nutrient medium or (ii) the bioreactor broth before or during syngas fermentation.

(10) The method of any one of aspects 1-9, wherein the at least one acetogenic carboxydotrophic bacterium is cultured in the bioreactor to produce an acetogenic carboxydotrophic bacteria culture.

(11) The method of aspect 10, wherein the acetogenic carboxydotrophic bacteria culture continuously produces at least one oxygenated product for more than about 24 hours.

(12) The method of any one of aspects 1-11, wherein the conditions within the bioreactor create a bioreactor broth and the at least one oxygenated product is extracted from the bioreactor broth.

(13) The method of any one of aspects 1-12, wherein the at least one oxygenated product is ethanol.

(14) The method of any one of aspects 1-13, wherein the at least one acetogenic carboxydotrophic bacterium is recombinant.

(15) The method of aspect 14, wherein the at least one acetogenic carboxydotrophic bacterium was engineered to include at least one gene selected from thiH, thiS, thiF, and thiG.

(16) A method for producing from a gaseous substrate comprising CO, CO₂, and optionally H₂ at least one oxygenated product, the method comprising:
(a) providing to a bioreactor: (1) a gaseous substrate comprising CO, CO₂, and optionally H₂, (2) at least one acetogenic carboxydotrophic bacterium, and (3) a liquid nutrient medium, and
(b) providing conditions within the bioreactor for the at least one acetogenic carboxydotrophic bacterium to convert the gaseous substrate to at least one oxygenated product, wherein the at least one acetogenic carboxydotrophic bacterium comprises at least one exogenous gene selected from thiH, thiS, thiF, and thiG.

(17) A method of using a broth deficient in thiamine or thiazole-containing thiamine precursors as a screening tool for suitable ethanol producing acetogenic carboxydotrophic bacteria strains, the method comprising:
(a) providing to a bioreactor in the absence of thiamine or thiazole-containing thiamine precursors: (1) a gaseous substrate comprising CO, CO₂, and optionally H₂, (2) at least one acetogenic carboxydotrophic bacterium, and (3) a liquid nutrient medium, and
(b) providing conditions within the bioreactor for the at least one acetogenic carboxydotrophic bacterium to convert the gaseous substrate to at least one oxygenated product,
wherein the conversion of the gaseous substrate to at least one oxygenated product indicates the presence of a suitable ethanol producing acetogenic carboxydotrophic bacteria strain.

(18) A method of controlling bacterial contamination in a bioreactor, the method comprising:
(a) providing to a bioreactor in the absence of thiamine or thiazole-containing thiamine precursors: (1) a gaseous substrate comprising CO, CO₂, and optionally H₂, (2) at least one acetogenic carboxydotrophic bacterium, and (3) a liquid nutrient medium, and
(b) providing conditions within the bioreactor for the at least one acetogenic carboxydotrophic bacterium to convert the gaseous substrate to at least one oxygenated product,
wherein the absence of exogenously supplied thiamine or thiazole-containing thiamine precursors in the bioreactor prevents contamination of the bioreactor broth with non-desirable acetogenic carboxydotrophic bacteria.

(19) The method of any one of claims 1-18, wherein the at least one acetogenic carboxydotrophic bacterium is effective for use as animal feed.

(20) The method of any one of claims 1-18, wherein the at least one acetogenic carboxydotrophic bacterium is effective for use as fertilizer.

(21) A method of preparing animal feed, the method comprising:
(a) providing to a bioreactor: (1) a gaseous substrate comprising CO, CO₂, and optionally H₂, (2) at least one acetogenic carboxydotrophic bacterium comprising at least one gene selected from thiH, thiS, thiF, and thiG, and (3) a liquid nutrient medium,
(b) providing conditions within the bioreactor for the at least one acetogenic carboxydotrophic bacterium to convert the gaseous substrate to at least one oxygenated product, and
(c) removing the at least one acetogenic carboxydotrophic bacterium from the bioreactor,
wherein the at least one acetogenic carboxydotrophic bacterium is effective for use as animal feed.

(22) A method of preparing fertilizer, the method comprising:
(a) providing to a bioreactor: (1) a gaseous substrate comprising CO, CO₂, and optionally H₂, (2) at least one acetogenic carboxydotrophic bacterium comprising at least one gene selected from thiH, thiS, thiF, and thiG, and (3) a liquid nutrient medium,
(b) providing conditions within the bioreactor for the at least one acetogenic carboxydotrophic bacterium to convert the gaseous substrate to at least one oxygenated product, and
(c) removing the at least one acetogenic carboxydotrophic bacterium from the bioreactor,
wherein the at least one acetogenic carboxydotrophic bacterium is effective for use as fertilizer.

(23) A method of preparing animal feed, the method comprising:
(a) providing to a bioreactor in the absence of thiamine or thiazole-containing thiamine precursors: (1) a gaseous substrate comprising CO, CO₂, and optionally H₂, (2) at least one acetogenic carboxydotrophic bacterium, and (3) a liquid nutrient medium,
(b) providing conditions within the bioreactor for the at least one acetogenic carboxydotrophic bacterium to convert the gaseous substrate to at least one oxygenated product, and
(c) removing the at least one acetogenic carboxydotrophic bacterium from the bioreactor,
wherein the at least one acetogenic carboxydotrophic bacterium is effective for use as animal feed.

(24) A method of preparing fertilizer, the method comprising:
(a) providing to a bioreactor in the absence of thiamine or thiazole-containing thiamine precursors: (1) a gaseous substrate comprising CO, CO₂, and optionally H₂, (2) at least one acetogenic carboxydotrophic bacterium, and (3) a liquid nutrient medium,
(b) providing conditions within the bioreactor for the at least one acetogenic carboxydotrophic bacterium to convert the gaseous substrate to at least one oxygenated product, and
(c) removing the at least one acetogenic carboxydotrophic bacterium from the bioreactor,
wherein the at least one acetogenic carboxydotrophic bacterium is effective for use as fertilizer.

### Example 1

This example demonstrates that acetogenic carboxydotrophic bacteria strain SB1 produces acetate and ethanol during fermentation in growth medium without thiamine or thiazole-containing thiamine precursors.

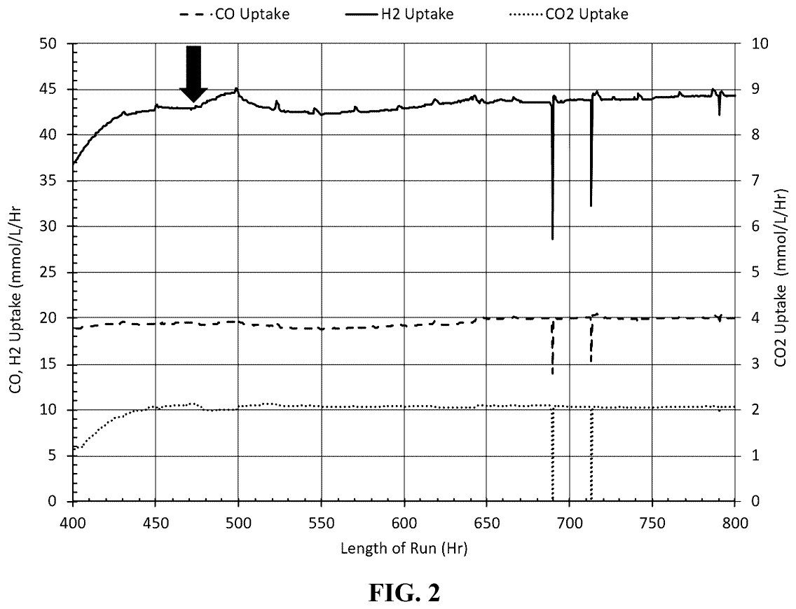

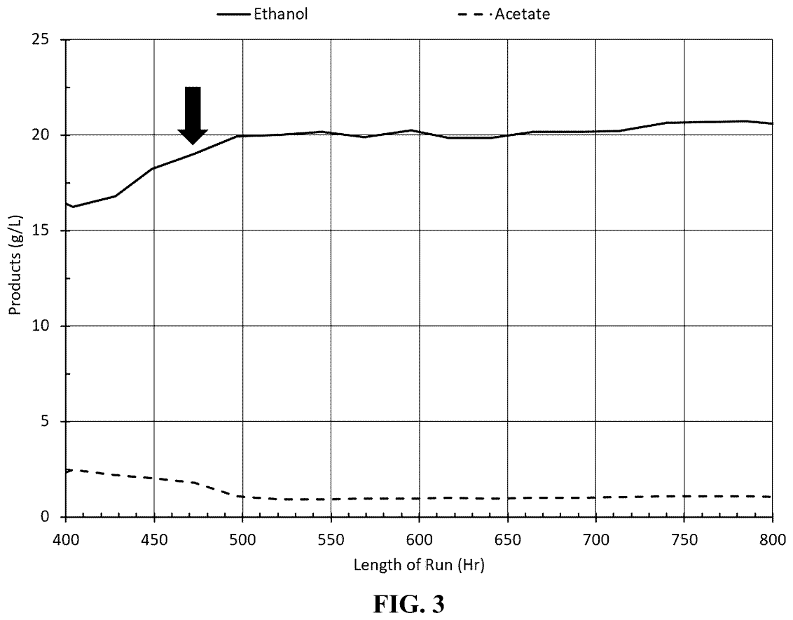

Fermentation was carried out in the presence of syngas (59% H₂, 25% CO, 2.7% CO₂, and 12.5% CH₄) using acetogenic carboxydotrophic bacteria strain SB1. At 473 hr, thiamine was removed from the growth medium. Growth continued for an additional 327 hr with no change in uptake of H₂, CO, or CO₂ (FIG. 2), and no decrease in production of ethanol or acetate (FIG. 3). Black arrows in FIGS. 2 and 3 denote 473 hr (when thiamine was removed). These results demonstrate that strain SB1 can undergo syngas growth without the addition of thiamine or thiamine precursors.

### Example 2

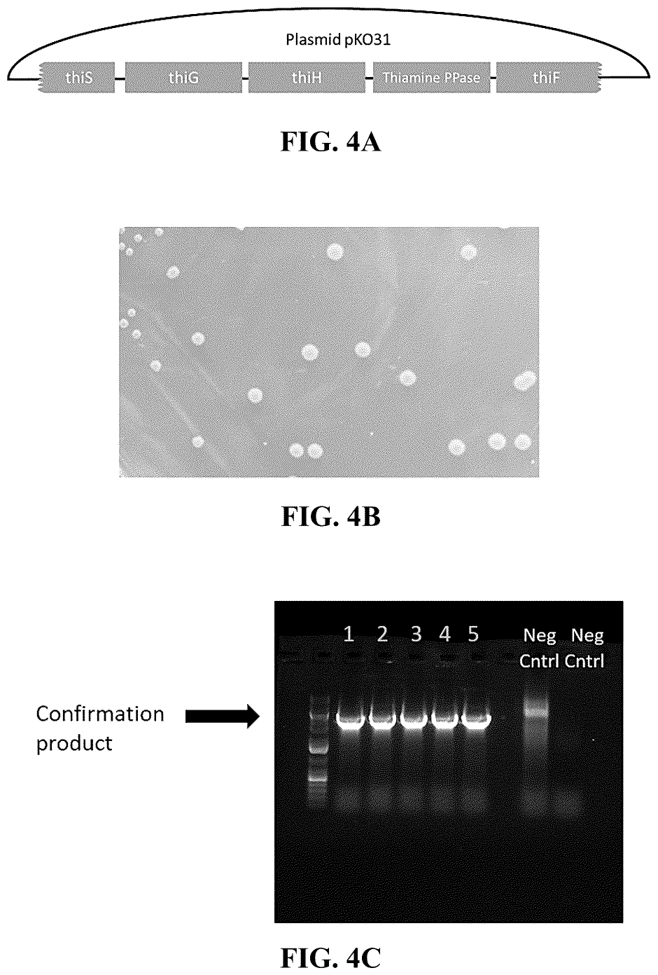

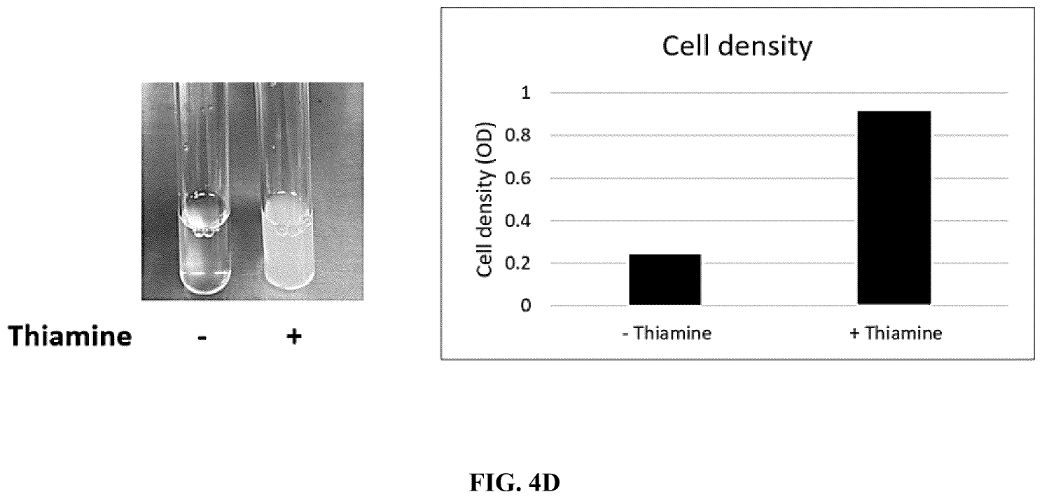

This example demonstrates that gene sequences SEQ ID NO: 1 (thiH), SEQ ID NO: 2 (thiS), SEQ ID NO: 3 (thiF), and SEQ ID NO: 4 (thiG) are required for the thiamine prototrophy of strain SB1. A mutant was constructed via single cross-over that was predicted to disrupt function of SEQ ID NO: 1 (thiH), SEQ ID NO: 2 (thiS), SEQ ID NO: 3 (thiF), and SEQ ID NO: 4 (thiG). A fragment was amplified via PCR that contained a 3,508 bp fragment spanning the region from nucleotide 3 of SEQ ID NO: 2 (thiS) to nucleotide 562 of SEQ ID NO: 3 (thiF). The fragment was cloned into a derivative of plasmid pMTL84151 (Heap, et al., Journal of Microbiological Methods, 78(1): 79-85 (2009)) to form plasmid pKO31 (FIG. 4A). Upon single cross-over recombination between the plasmid and the corresponding genes on the chromosome, the plasmid would be expected to integrate into the chromosome leading to disruption of the predicted thiHSFG thiazole biosynthetic genes.

Plasmid pKO31 was introduced via conjugation and single crossover integrations were selected using 2 ug/ml thiamphenicol to select for plasmid pKO31 and 10 ug/ml trimethoprim to counterselect against the E. coli donor strain. After conjugation, multiple antibiotic resistant colonies were isolated (FIG. 4B). The colonies were then screened for integration of the plasmid (FIG. 4C), and to demonstrate that the resulting colonies did not arise from tandem duplication of the entire region (data not shown).

Cells containing pKO31 integrated in the region of the predicted thiamine genes were then grown up in a Balch tube in fructose media (Mock, et al., J. Bacteriol., 197(18): 2965-2980 (2015)), and the cells washed to remove any thiamine in the media. The cells were then cultured in the same medium in the presence or absence of thiamine. Robust growth was only observed in the presence of thiamine for strain SB1 harboring the pKO31 integration (FIG. 4D; referred to as SB1::pKO31). Strain SB1 lacking the pKO31 insertion grew well in the absence of thiamine (FIGS. 2 and 3). The results demonstrate that disruption of the predicted thiazole biosynthetic genes lead to an inability to grow in media lacking thiamine.

### Example 3

This example demonstrates that gene sequences SEQ ID NO: 1 (thiH), SEQ ID NO: 2 (thiS), SEQ ID NO: 3 (thiF), and SEQ ID NO: 4 (thiG) are required for the thiamine prototrophy of strain SB1. A mutant (SB1::pKO31) was constructed via single cross-over that was predicted to disrupt function of SEQ ID NO: 1 (thiH), SEQ ID NO: 2 (thiS), SEQ ID NO: 3 (thiF), and SEQ ID NO: 4 (thiG) as described in Example 2.

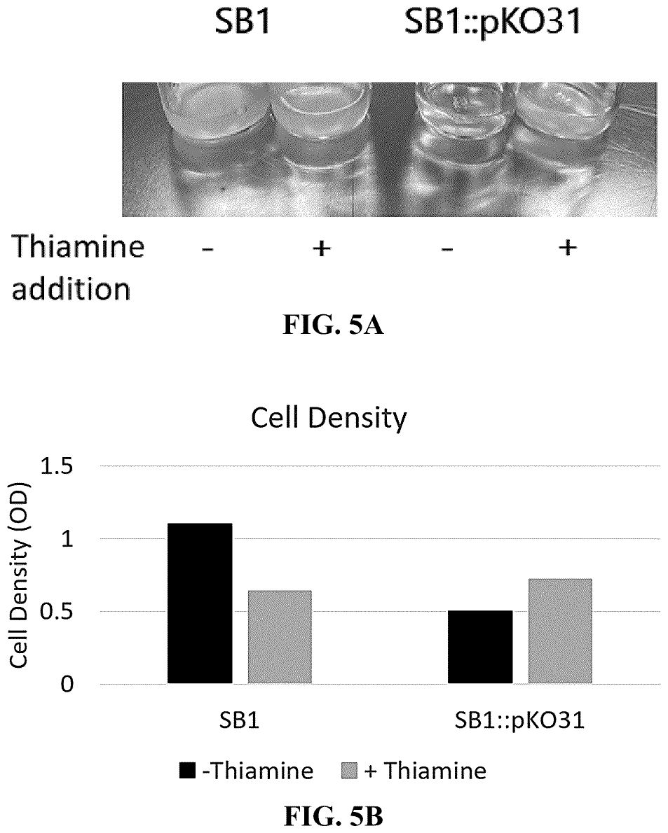

SB::pKO31 cells were then grown up in a bottle in fructose media (Mock, et al., J. Bacteriol., 197(18): 2965-2980 (2015)), and the cells washed to remove any thiamine in the media. The cells were then cultured in the same medium containing syngas in the presence or absence of thiamine (FIGS. 5A and 5B). Growth for SB1::pKO31 in syngas and thiamine was better than growth in syngas medium lacking thiamine. Strain SB1 showed robust growth in syngas medium in the presence and absence of thiamine. These results demonstrate that disruption of the predicted thiazole biosynthetic genes lead to an inability to grow in media lacking thiamine when syngas is used as a carbon source.

### Example 4

This example demonstrates that gene sequences SEQ ID NO: 1 (thiH), SEQ ID NO: 2 (thiS), SEQ ID NO: 3 (thiF), and SEQ ID NO: 4 (thiG) are required for the thiamine prototrophy of strain SB1. A mutant SB1::pKO31 was constructed via single cross-over that was predicted to disrupt function of SEQ ID NO: 1 (thiH), SEQ ID NO: 2 (thiS), SEQ ID NO: 3 (thiF), and SEQ ID NO: 4 (thiG) as described in Example 2.

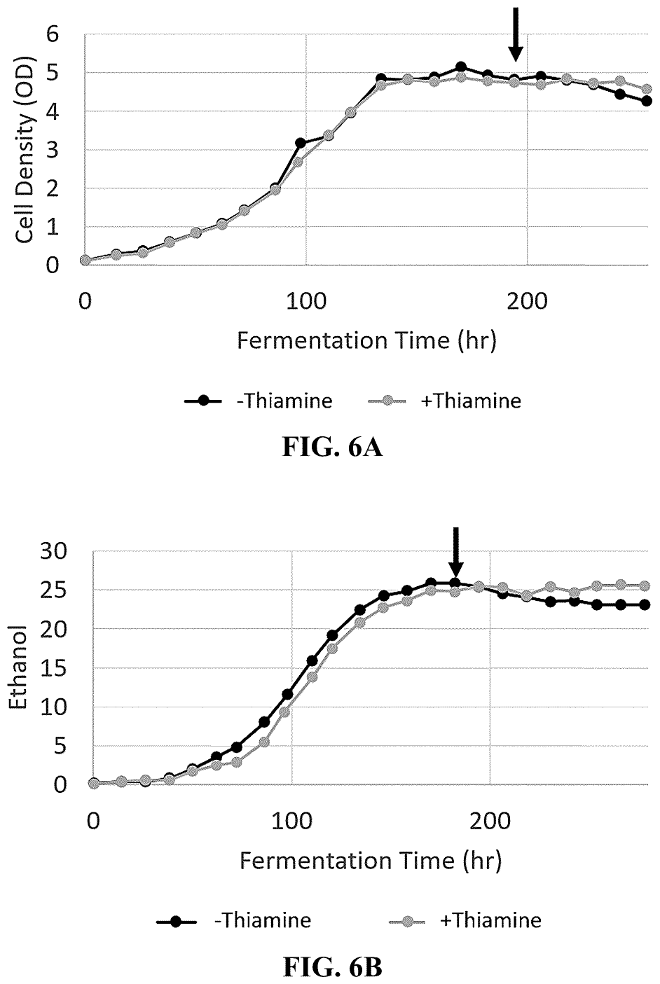

Cells containing SB1::pKO31 were then grown in fructose media (Mock, et al., J. Bacteriol., 197(18): 2965-2980 (2015)), and the cells used to inoculate a syngas reactor. The reactor was then allowed to reach steady state, as described in Example 1, with the addition of 5 ug/ml thiamphenicol (to select for maintenance of the integrated plasmid). At 185 hr (arrow) thiamine was removed from the media. Removal of thiamine from the media feed led to a small decline in optical density (OD600) (FIG. 6A), as well as in production of ethanol (FIG. 6B). The results demonstrate that disruption of the predicted thiazole biosynthetic genes lead to a reduced ability to grow in media lacking thiamine during steady state continuous fermentation in syngas. Removal of thiamine did not affect either growth or productivity of strain SB1 that lacked the integrated plasmid in the predicted thiazole biosynthesis genes (see Examples 1 and 2).

Although a reduction in growth and ethanol productivity was observed after removal of the thiamine from the media, the effect was less pronounced than what was observed in tube/bottle experiments with either fructose or syngas as a carbon source. The reason for this is unknown, but could be related to the nature of the mutation constructed (which may retain a low level of thiFGSH functionality), as well as the slow growth that accompanies steady state growth in a bioreactor (e.g., in fructose, bacteria growth is faster and therefore changes in growth rate are observed quicker).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments and aspects of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of producing from a gaseous substrate comprising CO, CO₂, and optionally H₂ at least one oxygenated product, the method comprising:
(a) providing to a bioreactor: (1) a gaseous substrate comprising CO, CO₂, and optionally H₂, (2) at least one acetogenic carboxydotrophic bacterium, and (3) a liquid nutrient medium, and
(b) providing conditions within the bioreactor for the at least one acetogenic carboxydotrophic bacterium to convert the gaseous substrate to at least one oxygenated product,
wherein the at least one acetogenic carboxydotrophic bacterium comprises at least one gene selected from thiH, thiS, thiF, and thiG.

2. The method of claim 1, wherein the at least one acetogenic carboxydotrophic bacterium comprises a sequence with at least 90% identity to SEQ ID NO: 1 (thiH).

3. The method of claim 1, wherein the at least one acetogenic carboxydotrophic bacterium comprises a sequence with at least 90% identity to SEQ ID NO: 2 (thiS).

4. The method of claim 1, wherein the at least one acetogenic carboxydotrophic bacterium comprises a sequence with at least 90% identity to SEQ ID NO: 3 (thiF).

5. The method of claim 1, wherein the at least one acetogenic carboxydotrophic bacterium comprises a sequence with at least 90% identity to SEQ ID NO: 4 (thiG).

6. The method of claim 1, wherein the at least one acetogenic carboxydotrophic bacterium comprises thiH, thiS, thiF, and thiG.

7. A method for producing from a gaseous substrate comprising CO, CO₂, and optionally H₂ at least one oxygenated product, the method comprising:
(a) providing to a bioreactor: (1) a gaseous substrate comprising CO, CO₂, and optionally H₂, (2) at least one acetogenic carboxydotrophic bacterium, and (3) a liquid nutrient medium, and
(b) providing conditions within the bioreactor for the at least one acetogenic carboxydotrophic bacterium to convert the gaseous substrate to at least one oxygenated product,
wherein the at least one acetogenic carboxydotrophic bacterium does not require exogenously supplied thiamine or thiazole-containing thiamine precursors.

8. The method of claim 1, wherein no more than 0.05 mg of thiamine or thiazole-containing thiamine precursors per 100 L is provided to the bioreactor.

9. A method of operating a syngas fermentation without the addition of thiamine or thiazole-containing thiamine precursors, the method comprising:
(a) providing to a bioreactor: (1) a gaseous substrate comprising CO, CO₂, and optionally H₂, (2) at least one acetogenic carboxydotrophic bacterium, and (3) a liquid nutrient medium that does not contain a thiamine or thiazole-containing thiamine precursors; and
(b) providing conditions within the bioreactor for the at least one acetogenic carboxydotrophic bacterium to convert the gaseous substrate to at least one oxygenated product,
wherein the conditions within the bioreactor create a bioreactor broth, and thiamine or thiazole-containing thiamine precursors is not added to (i) the liquid nutrient medium or (ii) the bioreactor broth before or during syngas fermentation.

10. The method of claim 1, wherein the at least one acetogenic carboxydotrophic bacterium is cultured in the bioreactor to produce an acetogenic carboxydotrophic bacteria culture.

11. The method of claim 10, wherein the acetogenic carboxydotrophic bacteria culture continuously produces at least one oxygenated product for more than about 24 hours.

12. The method of claim 1, wherein the conditions within the bioreactor create a bioreactor broth and the at least one oxygenated product is extracted from the bioreactor broth.

13. The method of claim 1, wherein the at least one oxygenated product is ethanol.

14. The method of claim 1, wherein the at least one acetogenic carboxydotrophic bacterium is recombinant.

15. The method of claim 14, wherein the at least one acetogenic carboxydotrophic bacterium was engineered to include at least one gene selected from thiH, thiS, thiF, and thiG.

16. A method for producing from a gaseous substrate comprising CO, CO₂, and optionally H₂ at least one oxygenated product, the method comprising:
(a) providing to a bioreactor: (1) a gaseous substrate comprising CO, CO₂, and optionally H₂, (2) at least one acetogenic carboxydotrophic bacterium, and (3) a liquid nutrient medium, and
(b) providing conditions within the bioreactor for the at least one acetogenic carboxydotrophic bacterium to convert the gaseous substrate to at least one oxygenated product,
wherein the at least one acetogenic carboxydotrophic bacterium comprises at least one exogenous gene selected from thiH, thiS, thiF, and thiG.


SEQUENCE LISTING (4 sequences)

SEQ ID NO: 1 - DNA, 1107 bp, synthetic construct
  source 1..1107: mol_type = other DNA
atgaccttttatgatgtagttcaaagtcttaaagactttgattatgacagcttttttaaaaatatttcagacatgcaaattcaacaaataattgctaaagataaattggacaagtttgattttttagccctactttctcctactgcagaaaatcacattgaagaaatggctcaaaaggcccacaacatttccttaaaatattttggcaaaaatgtacttatatataccccaatgtacgttgccaattattgctcaaatagatgtgcctactgcggatacaattgtacaaatgatataaacagaaaagttctatctcttgatgaagttgaaacagaagctaaagctgtacatgacaagggattcagacatgtaattcttttaacgggagaatctagaaaatactctcctgtttcctatataagggattgtgttaaaatactcaataaatatttcagttcaatatgccttgaaatctattctcttaaaaaagaagaatatgaagaacttgttaaagctggtgcagatagtttaaccatgtatcaggaaacctacaatgaggaagtatattcaaaggttcatcttggaggtccaaagaaaaattatagatatagacttgaaactccagaaagagcttgtgaagctggtatatactccgtaggtcttggtccactttatggtctttatgattggcgtgaggaagcttttctaggtggacttcacggtgcatacatccagaaaaagtttcccggagtagatgttaacttttcagttccaagaatacgccctcatgcaggatcatttaataatcttcacgaaataaacgagaaaactgtagttcaaatactactggcacttaaattatttattccaaggtcaggcaccaatataactacaagagaatgctcagaaatgagaaataacctcatacctcttggagtcactaaaatgtcagctggcgtatctacagcagttggaggccacactcaagaaaatccaggtgaaaagcaatttgatacatctgacaagagaagccttgaagaagttagagaagcaataacaaataagggctattttcctgtaatgaaagattgggaacccatgatacta

SEQ ID NO: 2 - DNA, 213 bp, synthetic construct
  source 1..213: mol_type = other DNA
atgaatataagagtaaatggagaaagcaaaagtataaaagaaggtgtaagcataacagaaattttaaaaattgaaaaagtaaaaatgccggatatggtttcagtacagctaaacgacgaatttgtaaacagaggcaattttaatagtacttttttgaaagaaaatgatatgatagaatttttgtacttcatgggaggtggtagtattgaattt

SEQ ID NO: 3 - DNA, 828 bp, synthetic construct
  source 1..828: mol_type = other DNA
ttgaattttagcgaggaacaaatagaaagatactcaagacatatactgttgtctgaagtaggagtagaaggtgaagaaaaacttttaaactctaaaattcttattataggaacaggaggacttggagcgcctgctgccatgtatctagccgctgccggaataggaaccataggacttgtagatggcgatgtagttgacctatcaaatcttcaaagacagataatacatgagaccaaagatgttgggaaatctaaagttttatcaggaaaagagactataaatagattgaatccggatgtaaatgtggtaacttacaacgaacttgttgattctcgcaatattttggacatcatacaagatcaaaactatgatttcataatagatggcacagacaatttccctgctaaatttttgataaatgatgcctgtgtactttctaaaaagcctttttctcacggtggaatactaagatttcaagggcagttaactacatacataccagacaatgggacaccatgttatagatgtattttccaatcgccgcctccagcaggcatagtacctacatgcagagaagccggtgttataggaattatgggtggaattatagggtcacttcaagcagctgaagcaatcaaatatttacttaaaattggcaatagccttgcagggtatcttttaacctataatgcaattaccatggaatttaggaaaataaaagtagccaaaaataaaaaatgtggaatatgcagcgatcatcctactataaaaaatcttatagattatgagcaggcagcttgtaacttgaaaacaggaagcttaagaggg

SEQ ID NO: 4 - DNA, 768 bp, synthetic construct
  source 1..768: mol_type = other DNA
atggatgaacttgtaatagcaaataaaaaaataaaaagcagatttttcattggaacagggaaattttcgtcaaatgaaatagttccagatattattaaaagctctgaagcacaggttataacagtagccttaagacgtatcgacatacatgctacagattcaagagataatatactgaattttatagataaagattgcatacttgtggcaaatacatccggtgcaagaaatgcagaggaagctatacgacttgctcatttagctaaagcaacaggatgtggaaattgggttaaaattgaggttatttcagatagcaaatatcttcttcctgataactatgaaactttaaaagctactgaagtacttgtaaaagaaggttttatagcacttccatatataagtccaaatttaatggacgcaaaaagacttgtagacgcaggtgcctctgcagtcatgcccttaggttctcctataggtacaaatagaggtcttaaaaccagggaacttttaaaaattctaattgatgaaatagacattcctgtagttgtagatgcaggtataggaaaaccctctcacgccgcagaagccatggaaatgggtgcagatgcagtacttgcaaatactgctcttgcaacagcaaaagatccagtactcatagctgaagctttcaaacttgcagtacaagcaggaagaaaagcctttctttcaaaacctggtattgaaagagaattggccagtgcttcatcaccactcacaggatttttaaga